(19) United States
(12) Patent Application Publication
(10) Pub. No.: US 2010/0226926 A1
(43) Pub. Date: Sep. 9, 2010

(54) Method of Detection of Fluorescence-Labeled Probes Attached to Diseased Solid Tissue

(75) Inventors: Gregory C. Loney, Los Altos, CA (US); Vikram Mohan, San Francisco, CA (US); Rob Monroe, San Carlos, CA (US); Bikash Sabata, San Jose, CA (US); Glenn Stark, Scotts Valley, CA (US)

Correspondence Address: WILSON, SONSINI, GOODRICH & ROSATI, 650 PAGE MILL ROAD, PALO ALTO, CA, 94304-1050, US

(73) Assignee: Bioimagene, Inc, Sunnyvale, CA (US)

(21) Appl. No.: 12/720,582

(22) Filed: Mar. 9, 2010

Related U.S. Application Data
(60) Provisional application No. 61/158,506, filed on Mar. 9, 2009.

(51) Int. Cl. A61K 39/395; C12Q 1/68

(52) U.S. Cl. 424/141.1; 435/6; 424/130.1

(57) ABSTRACT

Disclosed herein, in certain embodiments, is a method of treating breast cancer characterized by the amplification of HER2 genes in a subject in need thereof, comprising: (a) isolating a tissue sample comprised of a plurality of breast tumor cells; (b) isolating a first section from said tissue sample; (c) isolating a second section from an adjacent portion of said tissue sample; (d) contacting the first section with a first stain; (e) contacting the second section with a probe; (f) imaging the first section following contact with the stain to produce a first image; (g) analyzing the first image for abnormal microscopic features; (h) identifying areas of interest in the first image that display abnormal microscopic features; (i) electronically annotating the first image to identify the areas of interest; (j) imaging the second section following contact with the probe; (k) aligning the first image and the second image; and (l) analyzing areas of interest in the second image that correspond to an area of interest identified in the first image; wherein the subject is administered an anti-HER2 antibody if HER2 is amplified or providing an alternative treatment if HER2 is not amplified.

### CROSS-REFERENCE

This application claims the benefit of U.S. Provisional Application No. 61/158,506, filed Mar. 9, 2009, which application is incorporated herein by reference.

### BACKGROUND OF THE INVENTION

Approximately 25% to 30% of invasive breast cancers are characterized by the amplification and/or overexpression of HER2. Trastuzumab is a monoclonal antibody against HER2 that is administered to subjects that are confirmed to overexpress HER2.

The current gold-standard for specifying Herceptin treatment for patients diagnosed with breast cancer is a fluorescence based test marketed by Abbott Labs called HER2 Fluorescence In-situ Hybridization or FISH. The test looks for chromosomal abnormalities by analyzing one of the 46 pairs of human chromosomes. Chromosome 17 contains the HER2 gene. In normal cells, there are two chromosome 17 and therefore 2 HER2 genes.

The FISH analysis uses a fluorescence dye or marker to bind to the chromosome 17 (green) and another marker to bind to the HER2 gene (red). Another fluorescent dye is used to mark the cells nucleus (blue). Abnormal or cancerous cells can show a plurality of green signals, aneuploidy or plurality of red signals (gene amplification). Counting theses signals allow a pathologist to determine with high certainty whether the patient is a candidate for Herceptin treatment for breast cancer. The size of the nucleus is about 10 micron and the size of the fluorence probes are about 0.5 micron.

To accurately count and resolve closely spaced probes it is common to use a 60×, high NA (0.90) microscope objective. The microscopes are equipped with sensitive CCD cameras where the field of view at a 60× objective are on the order of 0.15 mm×0.15 mm. The stained breast tissue is placed on a slide that measures 25 mm×75 mm. Taking the top of the slide for a label area of 25 mm×25 mm, the area of the slide where stained tissue may reside is anywhere within a 25 mm×50 mm area. To search systematically for the fluorescent markers at 60× one would need to visit greater than 50,000 fields.

Accordingly, there remains a need for a method to search fluorescent stained solid-tissue sections efficiently and in a more automated way to save time and eliminate the drudgery of examining many fields of view.

### SUMMARY OF THE INVENTION

Disclosed herein in certain embodiments is a method of determining the amount of hybridization of a labeled probe, said method comprising: (a) isolating a biological sample comprised of a plurality of cells; (b) isolating a first section from said biological sample; (c) isolating a second section from an adjacent portion of said biological sample; (d) contacting the first section with a first stain; (e) contacting the second section with a labeled probe; (f) imaging the first section following contact with the stain to produce a first image; (g) identifying areas of interest in the first image based on microscopic features; (h) electronically annotating the first image to mark an area of interest; (i) imaging the second section following contact with the probe to produce a thumb nail image; (j) aligning the area of interest in the first image and the thumb nail image; (k) selecting fields of view of the thumb nail image for further imaging at a higher magnification based on alignment of annotations in the first image; (l) imaging selected fields of view in the thumb nail at a higher magnification; and (m) determining the amount of hybridization of the labeled probe based on the image of step (l) whereby the number of fields of view employed in determining the amount of hybridization of labeled probe is lower than the number of fields of view for determining the amount of hybridization of labeled probe in the absence of the alignment of step (j).

Disclosed herein, in certain embodiments, is a method of detecting the hybridization of a labeled probe, said method comprising: isolating a biological sample comprised of a plurality of cells; isolating a first section from said biological sample; isolating a second section from an adjacent portion of said biological sample; contacting the first section with a first stain; contacting the second section with a labeled probe; imaging the first section following contact with the stain to produce a first image; identifying areas of interest in the first image based on microscopic features; electronically annotating the first image to mark an area of interest; imaging the second section following contact with the probe; aligning the first image and the second image; analyzing the level of hybridization in an area of interest in the second image that correspond to an area of interest identified in the first image; and identifying the field of views that best convey the amount of hybridization. In some embodiments, the level of hybridization is analyzed with a computer program. In some embodiments, the biological sample is a tissue sample. In some embodiments, the tissue sample is a breast tissue sample. In some embodiments, the biological sample is encased in paraffin. In some embodiments, the stain facilitates identification of a neoplastic cell. In some embodiments, the first stain is a stain for microscopic features. In some embodiments, the first stain is a fluorescently-labeled dye, or a non-fluorescent dye. In some embodiments, the first stain is H&E. In some embodiments, the probe is a probe for microscopic structures. In some embodiments, the probe facilitates identification of a nucleic acid sequence of interest. In some embodiments, the probe hybridizes with a HER2 gene. In some embodiments, the probe is a fluorescently-labeled probe, or a radio-labeled probe. In some embodiments, the method further comprises contacting the second section with a second stain. In some embodiments, the second stain facilitates the identification of microscopic structures. In some embodiments, the second stain stains chromosomes. In some embodiments, the method further comprises contacting the second section with a third stain. In some embodiments, the third stain facilitates the identification of microscopic structures. In some embodiments, the third stain stains a nucleus. In some embodiments, the number of fields of view to be analyzed are reduced.

Disclosed herein, in certain embodiments, is a method of detecting the hybridization of a fluorescently-labeled probe, said method comprising: isolating a biological sample comprised of a plurality of cells; isolating a first section from said biological sample; isolating a second section from an adjacent portion of said biological sample; contacting the first section with a first stain; contacting the second section with a labeled probe; imaging the first section following contact with the stain to produce a first image; identifying areas of interest in the first image based on microscopic features; electronically annotating the first image to mark an area of interest; imaging the second section following contact with the probe; aligning the first image and the second image; analyzing the level of hybridization in an area of interest in the second image that correspond to an area of interest identified in the first image; and identifying the field of views that best convey the amount of hybridization

Disclosed herein, in certain embodiments, is a method of identifying a HER2 amplified biological sample, said method comprising: isolating a biological sample comprised of a plurality of tumor cells; isolating a first section from said biological sample; isolating a second section from an adjacent portion of said biological sample; contacting the first section with a first stain; contacting the second section with a probe that hybridizes to HER2; imaging the first section following contact with the stain to produce a first image; analyzing the first image for abnormal microscopic features; identifying areas of interest in the first image that display abnormal microscopic features; electronically annotating the first image to identify the areas of interest; imaging the second section following contact with the probe; aligning the first image and the second image; analyzing the level of hybridization in an area of interest in the second image that correspond to an area of interest identified in the first image; and identifying the field of views that best convey the amount of hybridization.

Disclosed herein, in certain methods, is a method of treating breast cancer characterized by the amplification of HER2 genes in a subject in need thereof, comprising: isolating a biological sample comprised of a plurality of breast tumor cells; isolating a first section from said biological sample; isolating a second section from an adjacent portion of said biological sample; contacting the first section with a first stain; contacting the second section with a probe; imaging the first section following contact with the stain to produce a first image; analyzing the first image for abnormal microscopic features; identifying areas of interest in the first image that display abnormal microscopic features; electronically annotating the first image to identify the areas of interest; imaging the second section following contact with the probe; aligning the first image and the second image; analyzing the level of hybridization in an area of interest in the second image that correspond to an area of interest identified in the first image; and identifying the field of views that best convey the amount of hybridization; wherein the subject is administered an anti-HER2 antibody if HER2 is amplified or providing an alternative treatment if HER2 is not amplified.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a graphic display of an imaging method disclosed herein

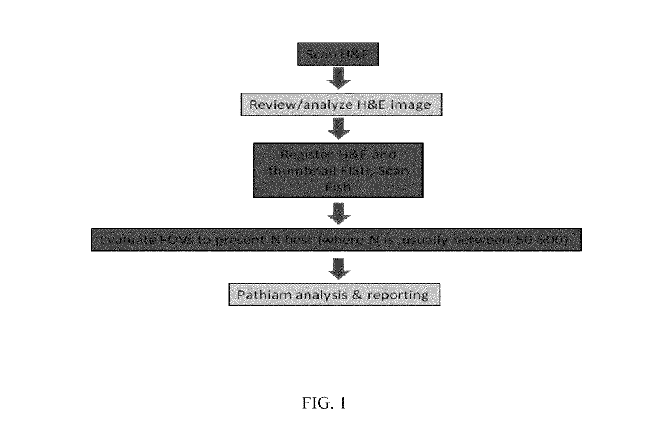

FIG. 2 demonstrates an H&E stained tissue section.

FIG. 3 demonstrates a marked-up (or annotated) H&E tissue section.

FIG. 4 demonstrates a phase contrast image of FISH stained tissue.

FIG. 5 demonstrates an H&E tissue image superimposed on a phase contrast FISH image.

FIG. 6 demonstrates an H&E image aligned with a phase contrast FISH image.

FIG. 7 demonstrates a stained HER2 FISH image.

### DETAILED DESCRIPTION OF THE INVENTION

### HER2

HER2 is a receptor tyrosine kinase found on chromosome 17 at locus 17q12-21.32. As there are two copies of chromosome 17 in each cell, there are also normally two copies of the HER2 gene. In certain instances, the binding of a ligand (e.g., epidermal growth factor, transforming growth factor a) to a HER2 partially or fully results in cell growth and differentiation.

Approximately 25% to 30% of invasive breast cancers are characterized by the amplification (i.e., the presence of more than two copies of HER2 ) and/or overexpression of HER2. Further, there is about a 90% correlation between HER2 amplification and the overexpression of HER2 receptor. In certain instances, the overexpression of HER2 receptor on a cell surface results in the abnormal proliferation of the cell. In certain instances, the aberrant expression of HER2 in breast cancer patients results in shortened disease-free survival (DFS) and poor clinical outcome.

### Trastuzumab

Trastuzumab is a monoclonal antibody against HER2. It is most effective in tumor cells that overexpress HER2. In certain instances, the binding of trastuzumab to HER2 ssuppresses HER2 activity. In certain instances, the binding of trastuzumab to HER2 decreases cell proliferation, induces cell stasis, and/or induces apoptosis. Due to the high cost of trastuzumab (it costs approximately $60,000 per annum per patient) and its selective efficacy, trastuzumab is only administered to subjects that are known to overexpress HER2.

### HER2 Analysis

HER-2 status is often determined by a two step process which is performed manually or semi-automated. Both the manual method and the semi-automated method begin by creating two sets of slides. The first set (the “morphological slide”) is designed for identification of abnormal tissue based on the color and morphology of cell structures (e.g., by staining with hematoxylin and eosin, aka H&E). In the second set is designed for analysis by fluorescence in situ hybridization (FISH).

The semi-automated method progresses as follows. First, the morphological slide is scanned into a computer system and areas of interest are identified. Second, the FISH slide is then imaged and the identified areas from the first slide are mapped to the FISH slide manually using an ink marker. Next, a pathologist manually drives a microscope to the areas of interest identified from the morphological slide and acquires images from this area. Finally, an algorithm analyzes the selected areas to determine whether HER2 is amplified.

The manual method progresses as follows. First, the morphological slide is viewed using a microscope and areas of interest are marked for FISH analysis. Second, the FISH slide is placed under the microscope and areas of interest are viewed under a higher magnification. Finally, a pathologist analyzes the selected areas to determine whether HER2 is amplified.

### FISH

In FISH, tissue samples are probed with fluorescently-labeled DNA sequences that are complementary to a gene of interest. With regards to FISH analysis of HER2, the probe is a sequence of DNA from the HER2 gene. In certain instances, the probe is constructed “in-house.” In certain instances, the probe is obtained from a commercial supplier (e.g., Vysis).

In certain instances, a 3-4 mm tissue section is cut from a tumor sample encased in paraffin. In certain instances, the tissue sample is prepared for FISH as follows. First, the tissue sample is deparaffinized by any suitable method (e.g., by washing in Hemo-De). Second, the tissue sample is dehydrated (e.g., by washing with ethanol) and air-dried. Third, the tissue sample is immersed in 0.2N HCl. Fourth, the tissue sample is treated with sodium thiocyanate solution. Proteins are removed from the tissue sample by use of protease digestion. Following protease digestion, the sample is fixed (e.g., with formalin). The sample is then denatured (e.g., with formamide/2×SSC solution) and dehydrated. Next, the sample is hybridized overnight. The sample is then washed to removed unbound and/or loosely bound probes. Finally, the sample is counterstained (e.g., with DAPI) and analyzed.

In certain instances, chromosomes are stained with fluorophores that fluoresce at a first wavelength (e.g., green) and the HER2 gene is stained with fluorophores (i.e., bound by a fluorophore-labeled probe) that fluoresce at a second wavelength (e.g., red or orange). In certain instances, a nucleus is stained with fluorophores that fluoresce at a third wavelength (e.g., blue). In certain instances, a tumor sample is positive for HER2 amplification if the ratio of HER2 to chromosome 17 is 2 or greater. In certain instances, a subject with a HER2:chromosome 17 ratio of 2 or higher is administered trastuzumab.

The current methods of analysis of these HER2 FISH slides are time-consuming, tedious and error prone. One method is fully manual and the other is semi-automated. In the fully manual method, all imaging is done using a microscope and the analysis is done by the human eye. In the semi-automated method, the image is digitized using either a scanner or a microscope with a camera, areas of interest are identified for analysis, and the analysis is performed by an algorithm. The art currently used in each method is described below for both UroVision and PathVision.

Again, in both the manual and semi-automated PathVision FISH Analysis the slide creation process is the same. Two slides are cut from a paraffin block. One slide is stained with H&E and the other is marked with two FISH probes. In semi-automated FISH analysis, the H&E slide is scanned into the system and areas of interest are identified for FISH analysis. The FISH slide is then imaged and the identified areas from the H&E slide are mapped to the FISH slide manually using an ink marker. The algorithm analyzes the selected areas and counts the FISH signals. With manual analysis, for example PathVision FISH analysis, the H&E slide is viewed using a microscope and areas of interest are marked for FISH analysis. The FISH slide is placed under the microscope and appropriate (marked) areas are viewed under a higher magnification. The analysis is done by counting the number of FISH signals present in the areas of interest. The analysis is presented to the pathologist for verification.

The slide creation processes for the fully manual and semi-automated UroVision FISH Analysis are the same. Urine cells are spun down from a urine sample. Two slides with the urine cells are prepared; one is marked with a pap stain and the other is marked with four FISH probes. In the semi-automated method, the pap stained slide is digitized. The FISH slide is then imaged with a 10× magnification to identify cells of interest; subsequently the appropriate number of cells is imaged at either 40× or 60× by the scanner. An algorithm is run to analyze the image and count specs. In the manual UroVision FISH Analysis, the pap stained slide is viewed using a microscope. The FISH slide is reviewed by the cytotechnician or FISH technician and the FISH signals are counted manually on the appropriate number of cells. After either analysis is performed, the pathologist verifies the analysis and sends back the results.

### Method

Disclosed herein in certain embodiments 1 is a method of determining the amount of hybridization of a labeled probe, said method comprising: (a) isolating a biological sample comprised of a plurality of cells; (b) isolating a first section from said biological sample; (c) isolating a second section from an adjacent portion of said biological sample; (d) contacting the first section with a first stain; (e) contacting the second section with a labeled probe; (f) imaging the first section following contact with the stain to produce a first image; (g) identifying areas of interest in the first image based on microscopic features; (h) electronically annotating the first image to mark an area of interest; (i) imaging the second section following contact with the probe to produce a thumb nail image; (j) aligning the area of interest in the first image and the thumb nail image; (k) selecting fields of view of the thumb nail image for further imaging at a higher magnification based on alignment of annotations in the first image; (l) imaging selected fields of view in the thumb nail at a higher magnification; and (m) determining the amount of hybridization of the labeled probe based on the image of step (l) whereby the number of fields of view employed in determining the amount of hybridization of labeled probe is lower than the number of fields of view for determining the amount of hybridization of labeled probe in the absence of the alignment of step (j).

Disclosed herein, in certain instances, is a computer implemented method for comparing a first tissue section and a second tissue section. In some embodiments, the first tissue section and the second tissue section are obtained from adjacent portions of a tissue sample. In some embodiments, the first tissue section is analyzed by a first method. In some embodiments, the first method is staining to identify microscopic features. In some embodiments, the first method is staining with a fluorescently-labeled dye, with a non-fluorescent dye, or radio-labeling. In some embodiments, the second tissue section is analyzed by a second method. In some embodiments, the second method is staining to identify microscopic structures (e.g., genes). In some embodiments, the second method is staining with a fluorescent dye, or radio-labeling. In some embodiments, the first section is used to identify abnormal cells. In some embodiments, the second section is used to identify a nucleic acid of interest (e.g., chromosome 17, or HER2).

Disclosed herein, in certain instances, is a method for searching fluorescent stained solid tissue sections. In some embodiments, a first tissue section on the order of 5 microns is cut from a tissue block. In some embodiments, a second section is cut in proximity to the first section and saved for FISH analysis.

In some embodiments, the first section is stained with H&E. In some embodiments, the first section is imaged using brightfield microscope optics. In some embodiments, the microscope magnification is 20×. In some embodiments, the microscope magnification is 40×.

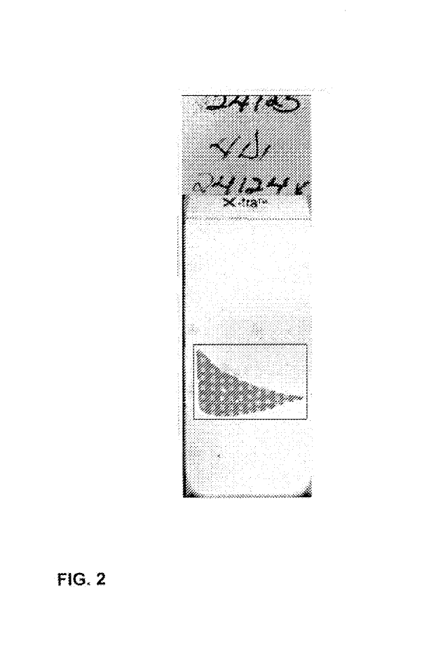

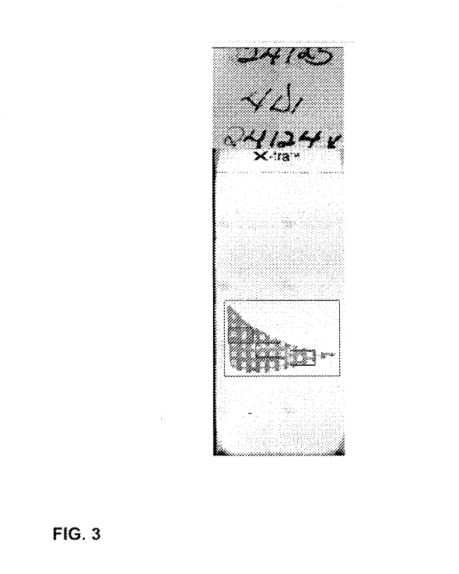

In some embodiments, the individual camera frames are assembled together to form a super-image which is analyzed (see FIG. 2). In some embodiments, color and morphology of cell structures are analyzed to identify abnormal or cancerous tissue. In some embodiments, the areas of the tissue that appear abnormal are marked (see FIG. 3). In some embodiments, the super-image shows an irregular shape which lends itself to a computerized centroid calculation and computation of principal axes. In some embodiments, this centroid and axes system allow the precise location of the marked abnormal sites to be referenced absolutely to the tissue blob. In some embodiments, the image, centroid and axes system, and the annotations are saved.

In some embodiments, the second section is subjected to FISH staining.

In some embodiments, the second section is imaged following FISH staining In some embodiments, the imaging of the second section begins with the identification of the edges of the FISH stained section.

In some embodiments identification of the tissue edges is accomplished by interrogating the

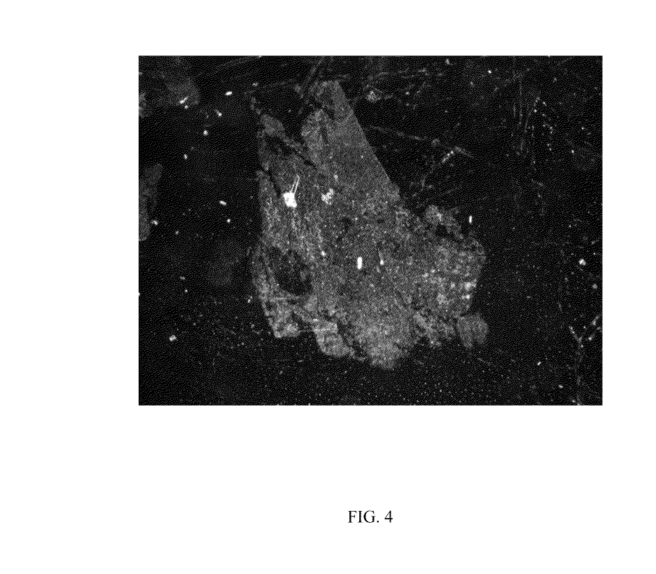

DAPI channel on a fluorescence microscope. DAPI is used to label cell nuclei and appears blue (emission wavelengths of 460-500 nm). In identifying the tissue edges a lower magnification can be used, for example through a 4× objective. The microscope fields of view are stitched together to provide an overall thumbnail view of the tissue. The DAPI stain is part of the protocol consistent with fluorescent in-situ hybridization (FISH). Combination of FISH and DAPI labeling allows the simultaneous detection of signals from DNA probes and the identification of nucleic location of the probe. In some embodiments, the edges of the FISH stained section are identified by phase-contrast microscopy, lowering the NA of the substage condenser by adjusting its iris, and/or the use of software to create phase contrast images from bright field mode microscopy. In some embodiments, a phase-contrast sub-stage condenser and a phase contrast microscope objective at 4× are used to determine the outline of the second section. An example of phase contrast FISH stained tissue at 10× is shown is FIG. 4.

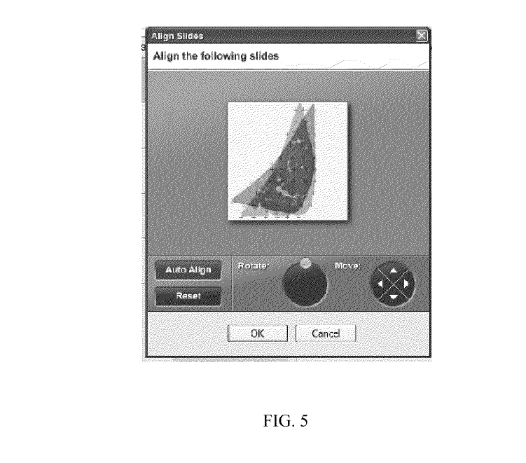

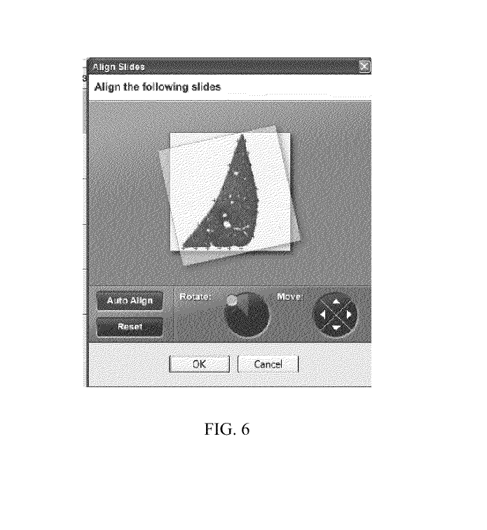

In some embodiments, the image obtained from the second section is placed over the image obtained from the first section (FIG. 5). In some embodiments, the first image and the second image are aligned to allow a coordinate mapping from one image to the other (FIG. 6). In some embodiments, the aforementioned centroiding and principal axes methods is used to align or register the H&E tissue and the FISH stained tissue.

In some embodiments, the areas of interest are subjected to further interrogation by a high magnification epi-flourescence microscope. In some embodiments, the magnifications are done at 40×. In some embodiments, the magnifications are done at 60×.

In some embodiments, the microscope stores image frames to blanket the areas of interest. In some embodiments, these frames are taken at different focus heights.

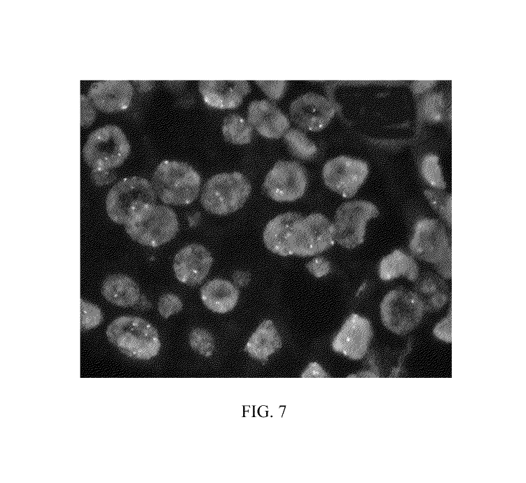

In some embodiments, the frames are analyzed by image processing software. In some embodiments, the software segments the cell nucleus and fluorescent probes of different color. FIG. 7 shows the cells and fluorescent. In some embodiments, the FISH probes are counted and scored by the software.

In an illustration of the methods disclosed herein and without limitation, automation of the above steps may be carried out through equipment and protocols such as but not limited to those available from Bioimagene Inc, (Sunnyvale Calif.). For example the iScan Concerto suite of products contains a brightfield and epi-fluorescence microscope optical train for allowing a user to combine both modes of imaging in one instrument. The iScan Concerto controller software registers the H&E image and FISH image and directs the XY and focus stages to the most likely places on the tissue as determined by the pathologist to find fluorescent tagged HER2 genes. The Virtuoso software analyzes the resulting FISH frames or fields of view (FOVs) to count probes and provide quantitative scores to the pathologist.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of determining the amount of hybridization of a labeled probe, said method comprising:
(a) isolating a biological sample comprised of a plurality of cells;
(b) isolating a first section from said biological sample;
(c) isolating a second section from an adjacent portion of said biological sample;
(d) contacting the first section with a first stain;
(e) contacting the second section with a labeled probe;
(f) imaging the first section following contact with the stain to produce a first image;
(g) identifying areas of interest in the first image based on microscopic features;
(h) electronically annotating the first image to mark an area of interest;
(i) imaging the second section following contact with the probe to produce a thumb nail image;
(j) aligning the area of interest in the first image and the thumb nail image of the second section;
(k) selecting fields of view of the thumb nail image for further imaging at a higher magnification based on alignment of annotations in the first image;
(l) imaging selected fields of view in the thumb nail at a higher magnification; and
(m) determining the amount of hybridization of the labeled probe based on the image of step (l) whereby the number of fields of view employed in determining the amount of hybridization of labeled probe is lower than the number of fields of view for determining the amount of hybridization of labeled probe in the absence of the alignment of step (j).

2. The method of claim 1 wherein the number of fields of view employed in determining the amount of hybridization of labeled probe is 10% or less of the number of fields of view for determining the amount of hybridization of labeled probe in the absence of the alignment of step (j).

3. The method of claim 1 wherein step (h) incorporates input from a human operator.

4. The method of claim 3 wherein the human operator is a pathologist.

5. The method of claim 4 wherein the pathologist indicates areas of interest through an electronic annotation tool wherein pathologist annotations are electronically stored.

6. The method of claim 1 wherein step (h), step (m) or both involve centroid calculations and/or computation of principal axes.

7. The method of claim 1, wherein the amount of hybridization is analyzed with a computer program.

8. The method of claim 1, wherein the biological sample is a tissue sample.

9. The method of claim 8, wherein the tissue sample is a breast tissue sample.

10. The method of claim 1, wherein the stain facilitates identification of a neoplastic cell.

11. The method of claim 1, wherein the first stain is a stain for microscopic features.

12. The method of claim 1, wherein the first stain is a fluorescently-labeled dye, or a non-fluorescent dye.

13. The method of claim 1, wherein the first stain is H&E.

14. The method of claim 1, wherein the probe is a probe for microscopic structures.

15. The method of claim 1, wherein the probe facilitates identification of a nucleic acid sequence of interest.

16. The method of claim 1, wherein the probe hybridizes with a HER2 gene.

17. The method of claim 1, wherein the probe is a fluorescently-labeled probe, or a radio-labeled probe.

18. The method of claim 1, further comprising contacting the second section with a second stain.

19. The method of claim 14, wherein the second stain facilitates the identification of microscopic structures.

20. The method of claim 14, wherein the second stain stains chromosomes.

21. The method of claim 1, further comprising contacting the second section with a third stain.

22. The method of claim 17, wherein the third stain facilitates the identification of microscopic structures.

23. The method of claim 17, wherein the third stain stains a nucleus.

24. The method of claim 1 wherein the probe is suitable for conducting fluorescence in-situ hybridization (FISH).

25. The method of claim 24 further comprising enhancing contrast of the second section thumbnail image by DAPI counterstaining or screening through phase contrast microscopy.

26. A method of detecting the hybridization of a labeled probe, said method comprising:
(a) isolating a biological sample comprised of a plurality of cells;
(b) isolating a first section from said biological sample;
(c) isolating a second section from an adjacent portion of said biological sample;
(d) contacting the first section with a first stain;
(e) contacting the second section with a labeled probe;
(f) imaging the first section following contact with the stain to produce a first image;
(g) identifying areas of interest in the first image based on microscopic features;
(h) electronically annotating the first image to mark an area of interest;
(i) imaging the second section following contact with the probe;
(j) aligning the first image and the second image;
(k) analyzing the level of hybridization in an area of interest in the second image that correspond to an area of interest identified in the first image; and
(l) identifying the field of views that best convey the amount of hybridization.

27. A method of detecting a the hybridization of a fluorescently-labeled probe, said method comprising:
(a) isolating a biological sample comprised of a plurality of cells;
(b) isolating a first section from said biological sample;
(c) isolating a second section from an adjacent portion of said biological sample;
(d) contacting the first section with a first stain;
(e) contacting the second section with a fluorescently labeled probe;
(f) imaging the first section following contact with the stain to produce a first image;
(g) identifying areas of interest in the first image based on microscopic features;
(h) electronically annotating the first image to mark an area of interest;
(i) imaging the second section following contact with the probe;
(j) aligning the first image and the second image;
(k) analyzing the level of hybridization in an area of interest in the second image that correspond to an area of interest identified in the first image; and
(l) identifying the field of views that best convey the amount of hybridization.

28. A method of identifying a HER2 amplified biological sample, said method comprising:
(a) isolating a biological sample comprised of a plurality of tumor cells;
(b) isolating a first section from said biological sample;
(c) isolating a second section from an adjacent portion of said biological sample;
(d) contacting the first section with a first stain;
(e) contacting the second section with a probe that hybridizes to HER2;
(f) imaging the first section following contact with the stain to produce a first image;
(g) analyzing the first image for abnormal microscopic features;
(h) identifying areas of interest in the first image that display abnormal microscopic features;
(i) electronically annotating the first image to identify the areas of interest;
(j) imaging the second section following contact with the probe;
(k) aligning the first image and the second image;
(l) analyzing the level of hybridization in an area of interest in the second image that correspond to an area of interest identified in the first image; and
(m) identifying the fields of view that best convey the amount of hybridization.

29. A method of treating breast cancer characterized by the amplification of HER2 genes in a subject in need thereof, comprising:
(a) isolating a biological sample comprised of a plurality of breast tumor cells;
(b) isolating a first section from said biological sample;
(c) isolating a second section from an adjacent portion of said biological sample;
(d) contacting the first section with a first stain;
(e) contacting the second section with a probe;
(f) imaging the first section following contact with the stain to produce a first image;
(g) analyzing the first image for abnormal microscopic features;
(h) identifying areas of interest in the first image that display abnormal microscopic features;
(i) electronically annotating the first image to identify the areas of interest;
(j) imaging the second section following contact with the probe;
(k) aligning the first image and the second image; and
(l) analyzing the level of hybridization in an area of interest in the second image that correspond to an area of interest identified in the first image;
(m) identifying the field of views that best convey the amount of hybridization; wherein the subject is administered an anti-HER2 antibody if HER2 is amplified or providing an alternative treatment if HER2 is not amplified; and
(n) administering to the patient an agent that suppresses HER2 activity.

30. The method of claim 29 wherein the agent that suppresses HER2 activity is an antibody.

31. The method of claim 30 wherein the antibody is trastuzumab.

32. The method of claim 29 wherein the agent that suppresses HER2 activity is Herceptin
